(19) United States
(12) Patent Application Publication
(10) Pub. No.: US 2024/0013397 A1
(43) Pub. Date: Jan. 11, 2024

(54) OPHTHALMOLOGIC IMAGE PROCESSING SYSTEM, OPHTHALMOLOGIC IMAGE PROCESSING DEVICE, AND STORAGE MEDIUM FOR STORING OPHTHALMOLOGIC IMAGE PROCESSING PROGRAM

(71) Applicant: NIDEK CO., LTD., Gamagori-shi (JP)

(72) Inventors: Norimasa SATAKE, Gamagori-shi (JP); Tetsuya KANO, Gamagori-shi (JP); Haruka UEMURA, Gamagori-shi, Aichi (JP); Masanori NAKANO, Gamagori-shi, Aichi (JP)

(21) Appl. No.: 18/345,275

(22) Filed: Jun. 30, 2023

(30) Foreign Application Priority Data
Jul. 5, 2022 (JP) 2022-108377

(51) Int. Cl. G06T 7/00; A61B 3/00; A61B 3/10; G16H 50/20

(52) U.S. Cl. CPC G06T 7/0014; A61B 3/0058; A61B 3/0025; A61B 3/102; G16H 50/20; G06T 2207/30041; G06T 2207/10101

(57) ABSTRACT

An ophthalmologic image processing system includes: an ophthalmologic imaging device that is configured to capture the ophthalmologic image; a display unit that is configured to display various types of medical information; and a control unit that is configured to control the display unit to display the various types of medical information. The control unit includes at least one processor programmed to: acquire the ophthalmologic captured by the ophthalmologic imaging device; generate the various types of medical information to be displayed on the display unit by performing a plurality of mutually different processes on the acquired ophthalmologic image; and control the display unit to sequentially display the various types of medical information upon generating each of the various types of medical information.

### CROSS REFERENCE TO RELATED APPLICATION

This application is based on, and claims the benefit of priority from, Japanese Patent Application No. 2022-108377 filed on Jul. 5, 2022. The entire disclosure of the above application is incorporated herein by reference.

### TECHNICAL FIELD

The present disclosure relates to an ophthalmologic image processing system and ophthalmologic image processing device which are configured to process data of ophthalmologic images that are images of a tissue of the subject eye, and a storage medium for storing an ophthalmologic image processing program executed by the ophthalmologic image processing device.

### BACKGROUND

Technology for generating useful medical information for diagnosis and the like by processing captured ophthalmologic images has been known. For example, a ophthalmic information processing device performs processing such as generation of two-dimensional map images, generation of two-dimensional charts, and generation of deviation map images for ophthalmologic images. The two-dimensional map image shows a two-dimensional distribution of the thickness of a specific layer in the fundus. The two-dimensional chart shows the average thickness of a specific layer in each of multiple regions. The deviation map image shows the difference between the subject eye data and a normative-database. The ophthalmic information processing device displays a report together with the generated medical information arranged on a display section. In recent years, technology has been proposed to generate captured ophthalmologic images with improved image quality and medical information such as information on a disease of the subject eye.

### SUMMARY

In a typical ophthalmologic image processing device, a report including multiple types of medical information based on ophthalmologic images is created and displayed after all the processes for generating the multiple types of medical information have been completed. Therefore, the user could not check any of the medical information during the time until all the processes for generating multiple types of medical information were completed. Thus, the user had to just wait during the processing time. Therefore, it would be very useful if multiple types of medical information generated by processing the ophthalmologic images could be more efficiently displayed.

One of objectives of the present disclosure is to provide an ophthalmologic image processing system, an ophthalmologic image processing device, and a storage medium for storing an ophthalmologic image processing program that are capable of displaying multiple types of medical information generated by processing an ophthalmologic image more efficiently.

In a first aspect of the present disclosure, an ophthalmologic image processing system is configured to process an ophthalmologic image that is an image of a tissue of a subject eye. The system includes: an ophthalmologic imaging device that is configured to capture the ophthalmologic image; a display unit; and a control unit that is configured to control the display unit. The control unit includes at least one processor programmed to: acquire the ophthalmologic image captured by the ophthalmologic imaging device; generate various types of medical information to be displayed on the display unit by performing a plurality of mutually different processes on the acquired ophthalmologic image; and control the display unit to sequentially display the various types of medical information upon generating each of the various types of medical information.

In a second aspect of the present disclosure, an ophthalmologic image processing device is configured to process an ophthalmologic image that is an image of a tissue of a subject eye. The device includes: a control unit including at least one processor programmed to: acquire the ophthalmologic image captured by an ophthalmologic imaging device; generate various types of medical information to be displayed on a display unit by performing a plurality of mutually different processes on the acquired ophthalmologic image; and control the display unit to sequentially display the various types of medical information upon generating each of the various types of medical information.

In a third aspect of the present disclosure, a non-transitory, computer readable, storage medium stores an ophthalmologic image processing program for processing an ophthalmologic image that is an image of a tissue of a subject eye. The program, when executed by at least one processor of an ophthalmologic image processing device, causes the at least one processor to perform: acquiring the ophthalmologic image captured by an ophthalmologic imaging device; generating various types of medical information to be displayed on a display unit by performing a plurality of mutually different processes on the acquired ophthalmologic image; and controlling the display unit to sequentially display the various types of medical information upon generating each of the various types of medical information

According to the ophthalmologic image processing system, ophthalmologic image processing device, and a storage medium storing ophthalmologic image processing program as described above, a user can efficiently understand multiple types of the medical information generated by processing a ophthalmologic image.

### BRIEF DESCRIPTION OF DRAWINGS

FIG. 1 is a block diagram showing a schematic configuration of an ophthalmologic image processing system.

FIG. 2 is an explanatory diagram for explaining an example of an ophthalmologic imaging method.

FIG. 3 is an explanatory diagram for explaining one example of multiple processes executed on an ophthalmologic image and medical information generated by the processes.

FIG. 4 is an example of a display screen of a monitor when some of multiple types of medical information on a subject eye are being generated.

FIG. 5 is one example of the display screen of the monitor when all the multiple types of the medical information on the subject eye are generated and displayed.

FIG. 6 is a flowchart of ophthalmologic image processing performed by the ophthalmologic image processing device.

FIG. 7 is an explanatory diagram for explaining data structure of a setting table.

FIG. 8 is a flowchart of a multi-step process performed during ophthalmologic image processing.

FIG. 9 is an explanatory diagram for explaining one example of a method of extracting pixel rows from a two-dimensional tomographic image step by step.

FIG. 10 is a flowchart of a specific region prioritized process performed during ophthalmologic image processing.

FIG. 11 is an explanatory diagram for explaining one example of a method of extracting an image of a specific region from a two-dimensional tomographic image.

FIG. 12 is a flowchart of a simplified process performed during the ophthalmologic image processing when no detailed examination is required.

### DESCRIPTION OF EMBODIMENTS

<Overview>

An ophthalmologic image processing device exemplified in this disclosure processes data of an ophthalmologic image which is an image of a tissue of the subject eye. The control unit of the ophthalmologic image processing device executes an image acquisition step, a medical information generation step, and a sequential display step. At the image acquisition step, the control unit acquires the ophthalmologic image captured by an ophthalmologic imaging device. At the medical information generation step, the control unit generates multiple types of medical information which will be eventually displayed in sequence on a display unit by executing multiple different processes on the ophthalmologic image. At the sequential display step, the control unit sequentially displays the generated medical information on the display unit each time each of the multiple types of medical information is generated during the medical information generation step.

According to the ophthalmologic image processing device in this disclosure, the generated medical information is sequentially displayed each time of the multiple types of medical information is generated. Therefore, even before all the generation processes of the medical information are completed, the user can grasp some of the medical information that have been already generated and displayed. As a result, the user's waiting time is reduced, and the multiple types of medical information can be more efficiently and easily recognized by the user.

All the multiple types of medical information being generated may be sequentially displayed each time each of the medical information is generated. Alternatively, only some of multiple types of medical information may be sequentially displayed each time the medical information is generated. In this case, other of medical information may be displayed on the display unit at the same time.

In an embodiment described below, an OCT device that is configured to capture a fundus will be described as a ophthalmologic imaging device. In other words, the ophthalmologic image in the following embodiment is an OCT image of the fundus captured by the OCT device. However, multiple types of medical information may be generated by processing an ophthalmologic image different from an OCT image of the fundus. For instance, the ophthalmologic image may include images captured by at least one of a scanning laser ophthalmoscope (SLO), a fundus camera, and a corneal endothelial cell microscope.

Devices executing the image acquisition step, the medical information generation step, and the sequential display step can be selected appropriately. For example, the control unit of a personal computer (hereinafter referred to as “PC”) may execute all the steps. That is, the control unit of the PC may acquire the ophthalmologic image from the ophthalmologic imaging device and execute processing on the acquired ophthalmologic image. Also, the control unit of the ophthalmologic imaging device may execute all the steps. Moreover, the control units of multiple devices (for example, an ophthalmologic imaging device and a PC) may together execute the image acquisition step, the medical information generation step, and the sequential display step.

The control unit may further execute a frame display step where multiple display frames each set for a respective type of the medical information are displayed on the display unit. In the sequential display step, the control unit may sequentially display one of types of the medical information in a corresponding display frame each time the medical information is generated. In this case, by checking the multiple display frames, the user can grasp the position where each medical information will be displayed even before the medical information is actually displayed. Therefore, the user can more efficiently grasp the contents of each medical information that is sequentially displayed.

In the frame display step, the control unit may add an in-process display image indicating that the medical information corresponding to the display frame is being generated until the medical information is actually displayed. Instead of, or in addition to, the in-process display image, an explanatory display image explaining the medical information in the display frame may be added to the corresponding display frame. In the case of executing the in-process display, the user can easily grasp that the medical information is being generated (i.e., to be displayed later) in the display frame. In the case of executing the explanatory display, the user can recognize an explanation about the medical information corresponding to the display frame even before the actual medical information is displayed. Thereafter, the user can make various judgments (such as diagnosis) based on the medical information to be displayed later. Thus, the user can more efficiently and easily grasp multiple types of the medical information.

The specific method for adding the in-progress display image and the explanatory display image to the display frame can be appropriately selected. For example, at least one of a progress indicator, an analog clock icon, and a hourglass icon indicating that the medical information is being processed may be added as the in-progress display image. In addition, an explanation indicating at least one of the types, characteristics, and ways of understanding of each medical information may be added as the explanatory display image. The in-progress display image and the explanatory display image may be added inside the display frame, or may be added at an outside position adjacent to the display frame.

At the frame display step, the controller may display, in the corresponding display frame, previously generated medical information about the same subject eye for which the ophthalmologic image is currently captured until the medical information is generated and displayed. In this case, the user can grasp the past medical information about the same subject eye while the medical information is being generated and displayed in the corresponding display frame. Therefore, the user can appropriately compare the past medical information with the newly generated medical information, enabling the user to more efficiently understand the medical information.

Here, there may be medical information that was previously generated and saved regarding the ophthalmologic image to be processed before the medical information generation step is executed (hereinafter referred to as “pre-generated information”). In such a case, the controller may execute a pre-generated information display step to display the pre-generated information on the display unit, regardless of the progress of the medical information generation step. In this case, the user can understand the pre-generated information displayed on the display unit even before all the medical information is generated during the medical information generation step. Therefore, the user can more efficiently understand the state of the subject eye, etc.

For example, the data of the ophthalmologic image to be displayed on the display unit may be generated by the ophthalmologic imaging device as the pre-generated information from the RAW data of the ophthalmologic image obtained by the ophthalmologic imaging device. For instance, if the ophthalmologic imaging device is an OCT device, at least one of tomographic image data, Enface image data, etc., can be generated as the pre-generated information. In this case, the control unit may skip the process of generating medical information that has already been generated as the pre-generated information at the medical information generation step. Or, the control unit may regenerate the same type of medical information with higher quality or accuracy than the pre-generated information, and display it on the display unit.

Moreover, the control unit may display a previously captured or pre-generated two-dimensional front view image (for example, a two-dimensional front view image of the fundus, etc.) of the same subject eye as the subject eye for which the ophthalmologic image being processed is currently captured, regardless of the progress of the generation process of medical information. After the generation process of a thickness map, which represents the two-dimensional distribution of the thickness of a particular layer, or a normal eye comparison map, which compares the two-dimensional distribution to a normal eye, has been completed, the control unit may display the generated map by superposing the generated map onto the two-dimensional front view image being displayed on the display unit. In this case, if the user can grasp the state of the subject eye only with the two-dimensional front view image that was displayed before the map is displayed, the user can proceed to the next operation without waiting for the map to be displayed. Therefore, the user can more efficiently grasp the state of the subject eye, etc. Note that the two-dimensional front view image is not necessarily limited to an image generated based on the ophthalmologic image being processed. For example, a two-dimensional front view image previously captured or generated by a device different from the device that captured the target ophthalmologic image (for example, a scanning laser ophthalmoscope (SLO), or a fundus camera, etc.) may be displayed on the display unit.

The control unit may display the pre-generated information in a display frame corresponding to the type of the pre-generated information. In this case, the user can easily identify the type of the displayed pre-generated information by the display frame in which the information is displayed.

Here, the pre-generated information may be simple medical information of lower quality or accuracy than the same type of medical information to be generated during the medical information generation step. In this case, during the medical information generation step, medical information of higher quality or accuracy than the pre-generated information may be generated. During the sequential display step, newly generated high-quality or high-accuracy medical information may be displayed in place of the pre-generated information displayed in the display frame. In this case, the user can grasp the pre-generated information until the high-quality or high-accuracy medical information is generated. Then, once the high-quality or high-accuracy medical information is generated, the user can grasp the newly generated high-quality or high-accuracy medical information. Therefore, the medical information can be more easily and accurately understood.

The control unit may further execute a specific disease identification step to identify at least one of multiple diseases as a specific disease. At the frame display step, the control unit may display the medical information of another subject who suffers from the specific disease in the corresponding display frame while the corresponding medical information is being displayed. In this case, the user can grasp the medical information of the other subject who has the specific disease until the medical information is displayed in the display frame. Therefore, the user can appropriately compare the newly generated and displayed medical information with the medical information of the other subject, thereby more efficiently understanding the medical information.

The specific method for identifying the specific disease can be appropriately selected. For instance, the user may input an instruction specifying the specific disease into the ophthalmologic image processing device by operating an operating unit. The control unit may identify the disease specified by the user as the specific disease. In this case, the medical information of another subject with the disease specified by the user is displayed in the display frame. Also, the control unit may identify the specific disease based on information related to the subject eye. The information regarding the subject eye includes, for instance, disease information entered in the subject eye's medical record, disease information determined based on past medical information of the subject eye, or disease-related information determined based on different images or examination results than the ophthalmologic image being currently processed. Additionally, the control unit may identify the specific disease of the subject eye by processing at least part of the ophthalmologic image. In this case, by inputting the ophthalmologic image into a mathematical model trained by a machine learning algorithm, the specific disease of the subject eye (for example, a disease likely present in the subject eye, etc.) may be identified. Furthermore, the control unit may identify the specific disease depending on the type of the ophthalmologic image.

The method of displaying the past medical information of the subject eye (hereinafter, referred to as “past information”) or the medical information of another subject having the specific disease (hereinafter, referred to as “similar case information”) in the display frame can also be appropriately chosen. For example, the control unit may switch between the past information/the similar case information and the new medical information generated based on the ophthalmologic image and display the selected one in the display frame according to the instructions input by the user after the new medical information was displayed in the display frame.

The ophthalmologic image obtained at the image acquisition step may be a three-dimensional tomographic image of the subject eye's tissue. The control unit may also execute a position input acceptance step that accepts an input of an instruction specifying the position at which the two-dimensional tomographic image to be displayed on the display unit is generated from the three-dimensional tomographic image. During execution of the medical information generation step, if an input of an instruction for specifying the position is accepted, the control unit may preferentially execute the process of generating the two-dimensional tomographic image at the specified position from the three-dimensional tomographic image as medical information with higher priority over other medical information generation processes. When the user wishes to urgently check a two-dimensional tomographic image at a specific position, the user will specify the position for the two-dimensional tomographic image to be displayed on the display unit. Therefore, the control unit preferentially executes the process of extracting and generating the two-dimensional tomographic image at the specified position over other medical information generation processes. This enables the control unit to adequately provide the medical information to the user while avoiding a decrease in the user's work efficiency.

The method of executing the process to generate a two-dimensional tomographic image at the specified position can also be appropriately chosen. For example, in addition to the process of extracting the two-dimensional tomographic image at the specified position from the three-dimensional tomographic image, the control unit may execute the process of improving the image quality of the extracted two-dimensional tomographic image. In this case, for instance, the control unit may improve the image quality of the two-dimensional tomographic image by additionally using multiple two-dimensional tomographic images taken at the specified position. Moreover, the control unit may use a mathematical model trained by a machine learning algorithm to improve the image quality to enhance the image quality of the extracted two-dimensional tomographic image.

The control unit may further perform an image extracting step for partially extracting, according to a predetermined rule, multiple pixels or pixel rows from the entire pixels or pixel rows constituting an ophthalmologic image. At the medical information generation step, the control unit may generate medical information by performing at least one of multiple processes on the extracted image obtained at the image extraction step. At the sequential display step, the control unit may display the generated medical information on the display unit once the medical information based on the extracted image is generated. When at least one process is executed on the extracted image, the amount of processes executed by the control unit is reduced as compared to when all processes are executed on the entire ophthalmologic image. Thus, waiting time for user is further reduced, and multiple types of the medical information can be more efficiently and easily understood by the user.

Note that the method of partially extracting, according to a predetermined rule, multiple pixels or pixel rows from the entire pixels or pixel rows constituting an ophthalmologic image can be chosen as appropriate. For example, the control unit may extract a set of pixels from the entire pixels constituting one ophthalmologic image according to a predetermined rule (for example, every N pixels). In this case, the extracted image is an image with reduced resolution from the pre-extracted image, and thus the amount of processing is appropriately reduced. Additionally, the control unit may extract a set of pixel from all pixel rows constituting one two-dimensional image according to a predetermined rule (for example, every N pixel rows). Examples of multiple pixel rows constituting a two-dimensional image include pixel rows extending in the direction of the optical axis of the light for capturing an ophthalmologic image (e.g., multiple A-scan images in the case of an OCT image). In this case, as a portion of multiple pixel rows constituting an ophthalmologic image are evenly removed, the amount of processing is appropriately reduced. Furthermore, a three-dimensional image is also formed of a set of multiple pixels and pixel rows. Therefore, the control unit may extract a portion of pixel rows from all two-dimensional images constituting a three-dimensional image, according to a predetermined rule (for example, every N two-dimensional images). In other words, the control unit may extract a portion of pixels or pixel rows from a three-dimensional image using two-dimensional images as units. In this case, since the data amount of the extracted image is reduced compared to the data amount of the pre-extracted image, the amount of processing is appropriately reduced.

In the medical information generation step, the control unit may execute the same process on at least a part of the remaining image having pixels or pixel rows that were not extracted and left behind at the image extraction step after the process on the extracted image has been completed. Then, the control unit may generate medical information based on both the extracted image and the remaining image. At the sequential display step, once the generation of the medical information based on the extracted image and the remaining image is completed, the control unit may display the generated medical information on the display unit. In this case, after the medical information based on the extracted image was displayed, the medical information based on the extracted image and the remaining image is displayed. That is, after the medical information based on the extracted image was displayed first, high-quality medical information based on both the extracted image and the remaining image is displayed. Therefore, users can more efficiently and easily grasp the medical information.

At the medical information generation step, the control unit may perform processing on the entire remaining image, thereby performing processing on the entire ophthalmologic mage including the extracted image and the remaining image. In this case, one type of the medical information is generated by a two-step process, namely, processing on the extracted image and processing on the entire remaining images. Moreover, at the medical information generation step, after processing on the extracted image is completed, the control unit may perform processing on a part of the remaining image. In this case, the control unit may separately perform processing on the remaining image several times to display the medical information in a step-by-step manner.

The control unit may also execute a detailed examination determination step for determining whether a detailed examination is necessary for the ophthalmologic image. If the control unit determines that the detailed examination is necessary for the ophthalmologic image, the control unit may execute the medical information generation step without performing the image extracting step. For the ophthalmologic image that is determined by the control unit not to require the detailed examination, the control unit may execute the image extracting step and perform at least one of the multiple processes on the extracted image. In this case, high-quality medical information is generated for the ophthalmologic image that is determined to require the detailed examination, whereas the medical information is generated in a short time for the ophthalmologic image that is determined not to require the detailed examination. Therefore, the processing can be performed more efficiently.

The method for determining whether a detailed examination on the ophthalmologic image is necessary can be appropriately selected. For example, the control unit may calculate the probability of a disease existing in the tissue depicted in the ophthalmologic image by processing at least apart of the obtained ophthalmologic image. Then, the control unit may determine whether a detailed examination on the ophthalmologic image is necessary by determining whether the calculated probability exceeds a threshold value. In this case, the control unit may obtain the probability of a disease existing by inputting at least a part of the ophthalmologic image into a mathematical model trained by a machine learning algorithm. Examples of this mathematical model include models trained to output probability of a disease existing therein. The control unit may obtain the probability of a disease existing therein by performing image processing on at least a part of the ophthalmologic image. Also, the control unit may determine that a detailed examination on the ophthalmologic image is necessary if information related to the subject eye indicates a high probability of disease existing. Examples of information related to the subject eye include disease information entered into the medical record on the subject eye, disease information determined based on past medical information on the subject eye, or disease information determined based on images or test results other than the captured ophthalmologic image.

The control unit may further execute a specific region extracting step extracting a part of the image area within the ophthalmologic image as a specific region. At the medical information generation step, the control unit may generate medical information by executing at least one of the multiple processes on the specific region. At the sequential display step, the generated medical information may be displayed on the display unit each time the medical information based on the specific region is completed. Moreover, after processing on the specific region is completed, the control unit may perform the same process on at least a part of the area within the image area of the ophthalmologic image that is a remaining area other than the specific region. Then, the control unit may generate and display the medical information based on the specific region and the remaining area. In this case, since the medical information about the specific region is displayed quickly, it becomes easier for users to grasp the medical information more efficiently.

The method for setting the specific region within the image area of the ophthalmologic image can also be appropriately selected. For example, the control unit may set a region with a high probability of a disease existing within the image area of the ophthalmologic image as the specific region by processing at least a part of the obtained ophthalmologic image. In this case, the control unit may input at least one part of the ophthalmologic image into a mathematical model trained by a machine learning algorithm to obtain a region with a high probability of a disease existing therein. As for this mathematical model, examples include a mathematical model trained to output a region with a high probability of a disease existing therein. Moreover, the control unit may set the specific region based on the information related to the subject eye. Examples of information related to the subject eye include disease information entered into the medical record of the subject eye, disease information determined based on past medical information of the subject eye, or disease information determined based on images or test results different from the ophthalmologic image under examination.

The control unit may further execute a processing order setting step for setting the order of performing multiple processes on the ophthalmologic image during the medical information generation step in response to an instruction input by the user. In this case, the multiple processes performed on the ophthalmologic image are executed in the order instructed by the user, and the generated medical information is sequentially displayed on the display unit. Therefore, since the medical information the user wishes to confirm first is displayed first on the display unit, it becomes easier for users to grasp the medical information more efficiently.

It should be noted that, among the generation processes of various types of medical information, there is a process using medical information generated by another medical information generation process (referred to as an other-information using process). In such cases, at the medical information generation step, the control unit may first execute the generation process of medical information that will be used in the other-information using process before performing the other information using process. In this case, by the time the other information using process is executed, the medical information to be used in the other information using process has already been generated. Therefore, multiple types of medical information are generated more appropriately and displayed on the display unit.

### Embodiment

Next, one typical embodiment of the present disclosure will be described. The present embodiment illustrates processing ophthalmologic images of a fundus tissue of a subject eye E captured by an OCT device which is an ophthalmologic imaging device. The ophthalmologic images include, for example, three-dimensional tomographic images, two-dimensional tomographic images, and OCT angiographic images. However, the ophthalmologic images to be processed may be images of tissues other than the fundus tissue. For example, the target ophthalmologic images may be images of tissues other than the fundus of the subject eye E (for example, an anterior segment of the subject eye). Furthermore, images of biological tissues other than the subject eye E (for example, skin, digestive organs, or brain) may also be processed. As described above, the device capturing the images is not necessarily limited to the OCT device.

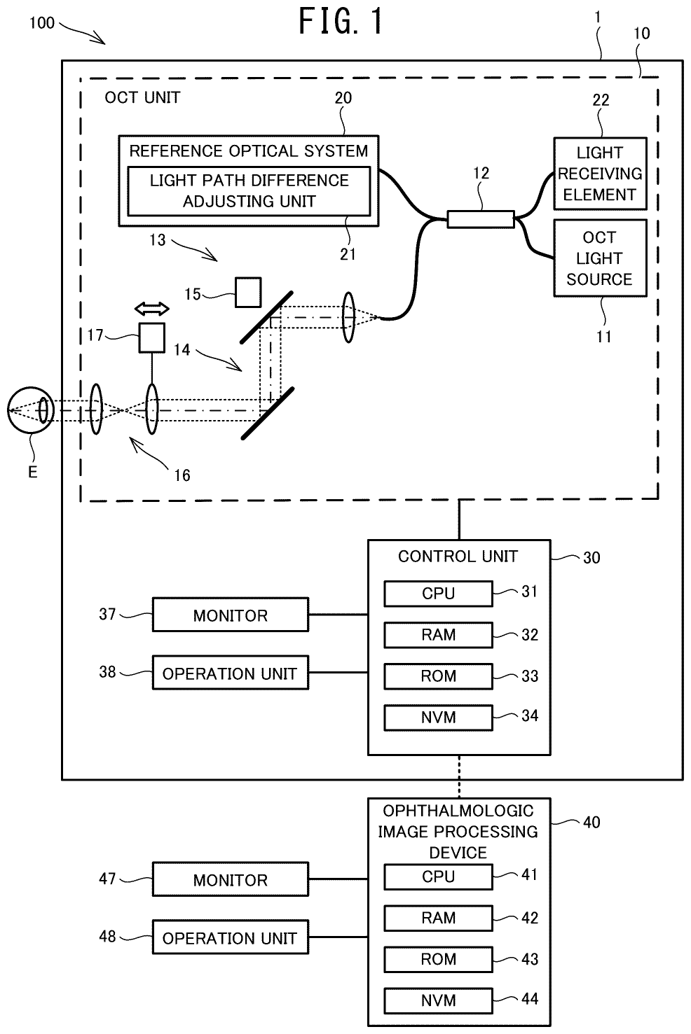

Referring to FIG. 1, a schematic configuration of the ophthalmologic image processing system 100 according to the present embodiment will be described. The ophthalmologic image processing system 100 in the present embodiment includes an ophthalmologic imaging device 1 and an ophthalmologic image processing device 40. The ophthalmologic imaging device 1 captures images (ophthalmologic images) of a living body (in this embodiment, the fundus of the subject eye). In detail, the ophthalmologic imaging device (OCT device) 1 of the present embodiment performs scanning with a measuring light on the tissue of a living body and continuously receives light from the tissue. Accordingly, a two-dimensional image that spreads in a first direction (a scanning direction) and a second direction (a depth direction of the optical axis of the measurement light) intersecting the first direction. Moreover, the ophthalmologic imaging device 1 emits the measurement light along each of multiple scanning lines within a two-dimensional measurement area in the living body. Accordingly, by capturing multiple two-dimensional tomographic images, a three-dimensional tomographic image of the living body can be obtained. Furthermore, the ophthalmologic imaging device 1 can obtain OCT angiographic data by performing scanning with the measuring lights emitted multiple times at the same position in the living body. OCT angiographic data is motion contrast data. Motion contrast data is generated by performing an arithmetic process on at least two OCT signals obtained at different times for the same position in the living body. The ophthalmologic image processing device 40 executes processing for the data of the ophthalmologic image captured (acquired) by the ophthalmologic imaging device 1.

Next, a configuration of the ophthalmologic imaging device 1 will be described. The ophthalmologic imaging device (OCT device) 1 is equipped with an OCT unit 10 and a control unit 30. The OCT unit 10 includes an OCT light source 11, a coupler (optical splitter) 12, a measuring optical system 13, a reference optical system 20, and a light-receiving element 22.

The OCT light source 11 emits light (OCT light) to acquire data of ophthalmologic images. The coupler 12 splits the OCT light emitted from the OCT light source 11 into measurement light and reference light. Also, in this embodiment, the coupler 12 multiplexes the measurement light reflected by the tissue (the fundus of the subject eye E in this embodiment) and the reference light generated by the reference optical system 20 to interfere with each other. In other words, the coupler 12 in this embodiment serves both as a branching optical element that splits the OCT light into the measurement light and the reference light and as a multiplexing optical element that multiplexes the reflected light of the measurement light and the reference light. Note that the structure of at least one of the branching optical element and the multiplexing optical element may be modified. For example, elements other than the coupler (for example, a circulator, a beam splitter, etc.) may be used.

The measurement optical system 13 introduces the measurement light split by the coupler 12 into the subject and returns the measurement light reflected by the tissue to the coupler 12. The measurement optical system 13 includes a scanning unit (scanner) 14, an irradiation optical system 16, and a focus adjusting unit 17. The scanning unit 14 is configured to scan a subject with a spot-like measurement light in a two-dimensional direction crossing the optical axis of the measurement light by being driven by the driving unit 15. In this embodiment, two galvanometer mirrors that polarize the measurement light to different directions respectively are used as the scanning unit 14. However, another device (for example, at least one of polygon mirror, resonant scanner, acoustic optical element and the like) that polarizes light may be used as the scanning unit 14. The irradiating optical system 16 is disposed at a downstream side of the scanning unit 14 (i.e., on a near side of the subject) in a light path to emit the measurement light on a tissue. The focus adjusting unit 17 moves an optical member (for example, lens) of the irradiating optical system 16 in a direction along the optical axis of the measurement light to adjust focus of the measurement light.

The reference optical system 20 generates the reference light and returns the reference light to the coupler 12. The reference optical system 20 of the present embodiment generates the reference light by reflecting the reference light branched by the coupler 12 using a reflection optical system (for example, a reference mirror). However, the structure of the reference optical system 20 may be also modified. For example, the reference optical system 20 may allow the light incident from the coupler to pass through the system 20 without reflecting the light and then return the light to the coupler 12. The reference optical system 20 includes a light path difference adjusting unit 21 that changes a difference between alight path of the measurement light and alight path of the reference light. In the present embodiment, the reference mirror is moved in the optical axis to change the difference between the light paths. A component that changes the difference between the light paths may be disposed in the light path of the measurement optical system 13.

The light receiving element 22 receives the interference light of the measurement light and the reference light generated by the coupler 12 to detect an interference signal. The present embodiment adopts a principle of Fourier domain OCT In the Fourier domain OCT spectrum intensity (spectrum interference signal) of the interference light is detected by the light receiving element 22. Then, a plurality of OCT signals are acquired through the Fourier transform of the spectrum intensity data. As one example of the Fourier domain OCT Spectral-domain-OCT (SD-OCT), Swept-source-OCT (SS-OCT) or the like can be adopted. Further, for example, Time-domain-OCT (TD-OCT) can be also adopted.

The control unit 30 controls the ophthalmologic imaging device 1. The control unit 30 includes a CPU 31, a RAM 32, a ROM 33, and a non-volatile memory (NVM) 34. The CPU 31 is a controller to perform a variety of controls. The RAM 32 temporarily stores various types of information. The ROM 33 stores programs executed by the CPU 31, various initial values, and the like. The NVM 34 is a non-transitory storage medium that is configured to keep the stored data even after the power is off.

A monitor 37 and an operation unit 38 are connected to the control unit 30. The monitor 37 is one example of a display unit that displays various images. The operation unit 38 is operated by a user for inputting various instructions of the user into the ophthalmologic imaging device 1. For example, various devices such as a mouse, a keyboard, a touch panel, and afoot switch can be used as the operation unit 38. The various instructions may be input into the ophthalmologic imaging device 1 as a sound input via a microphone.

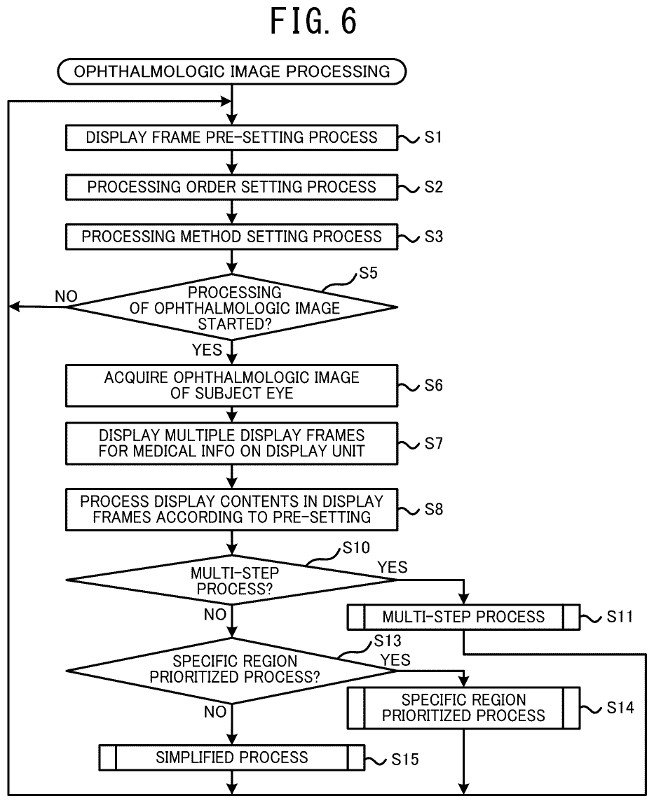

A schematic configuration of the ophthalmologic image processing device 40 will be described. In the present embodiment, a personal computer (hereinafter, referred to as a “PC”) is adopted as the ophthalmologic image processing device 40. However, a device other than the PC may be used as the ophthalmologic image processing device. For example, the ophthalmologic imaging device 1 itself may serve as the ophthalmologic image processing device that performs ophthalmologic image processes which will be described later. The ophthalmologic image processing device 40 is provided with a CPU 41, a RAM 42, a ROM 43, and an NVM 44. The CPU 41 is a controller to perform a variety of controls. Each of the RAM 42, the ROM 43, and the NVM 44 temporarily stores various information as described above. An ophthalmologic image processing program for performing an ophthalmologic image process (see FIG. 6) described below may be stored in the NVM 44. Further, a monitor 47 and an operation unit 48 are connected to the ophthalmologic image processing device 40. The monitor 47 is one example of a display unit that displays various images. The operation unit 48 is operated by a user for inputting various instructions of the user into the ophthalmologic image processing device 40. Similar to the operation unit 38 of the ophthalmologic imaging device 1, various devices such as a mouse, a keyboard, and a touch panel can be adopted as the operation unit 48. Further, the various instructions may be input into the ophthalmologic image processing device 40 as a sound input via a microphone.

The ophthalmologic image processing device 40 acquires various data (for example, data of an ophthalmologic image captured by the ophthalmologic imaging device 1, or the like) from the ophthalmologic imaging device 1. The various data may be acquired through, for example, at least one of wired-communication, wireless-communication, a detachable storage medium (for example, USB memory) and the like.

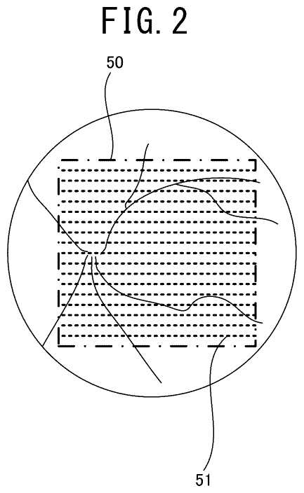

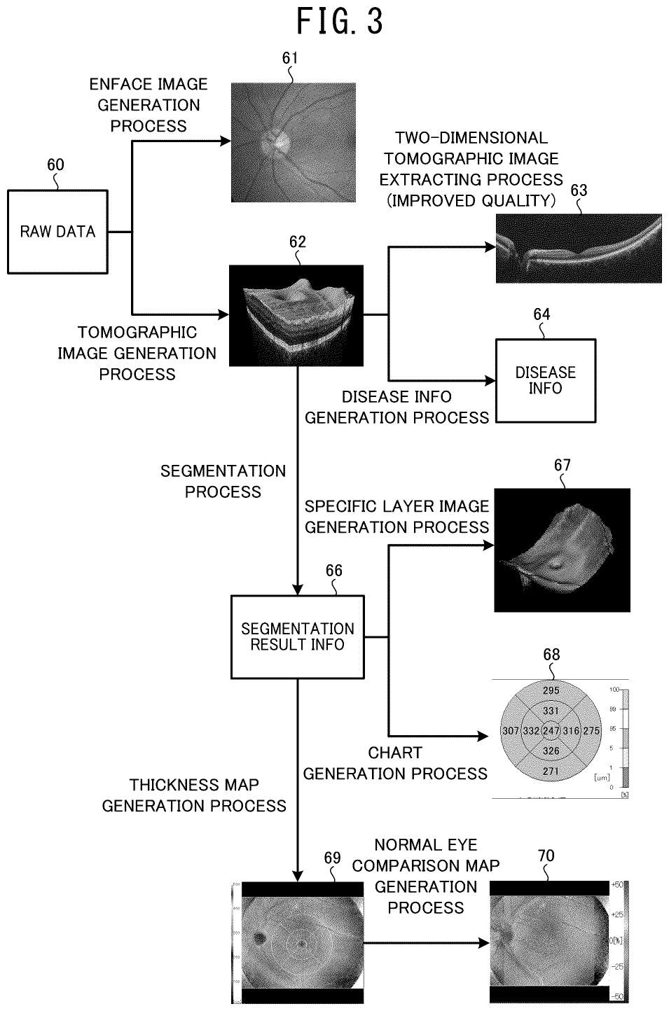

Referring to FIGS. 2 and 3, a method for capturing ophthalmologic images (a method for acquiring data of ophthalmologic images) by the ophthalmologic imaging device 1 according to the present embodiment and one example of medical information generated based on the ophthalmologic images will be described below. As shown in FIG. 2, in the ophthalmologic imaging device 1 according to the present embodiment, a two-dimensional measurement area 50 spreading in directions crossing the optical axis of OCT measurement light is set. Then, a plurality of linear scanning lines (scanning lines) 51 each of which scans a subject with a spot within this measurement area 50 is set at equal intervals. The ophthalmologic imaging device 1 scans a subject with a spot of the measurement light along a single scanning line 51. Accordingly, RAW data 60 for obtaining a two-dimensional tomographic image 63 (see FIG. 3) spreading from the scanning line 51 in the depth direction of the tissue is acquired (captured). The RAW data 60 is data itself of the ophthalmologic image captured and acquired by the ophthalmologic imaging device 1 (that is, raw data prior to subject to various processes). Also, the ophthalmologic imaging device 1 can scan a subject with the measurement light multiple times along the same scan line 51. Thus, the ophthalmologic imaging device 1 can also acquire RAW data 60 for obtaining an OCT angiography image or an addition average image. An addition average image is an image created by adding and averaging pixel values at the same position between multiple images. By performing addition averaging processing, noise in the image is reduced and an image quality improves. Also, the ophthalmologic imaging device 1 can scan a subject with a spot of the measurement light along each of multiple scanning lines 51. Thus, the ophthalmologic imaging device 1 can acquire (capture) RAW data 60 for obtaining a three-dimensional tomographic image 62 (see FIG. 3) of the tissue.

Referring to FIG. 3, an example of multiple processes performed on the ophthalmologic images to generate multiple types of medical information will be described. The ophthalmologic image processing device 40 according to the present embodiment performs multiple different processes on the ophthalmologic images captured by the ophthalmologic imaging device 1. Accordingly, the ophthalmologic image processing device 40 can generate multiple types of medical information. The ophthalmologic image may be the RAW data 60, which is the image data itself captured by the processing device 40 or data of images generated from the RAW data 60. The ophthalmologic image processing device 40 displays at least some types of the generated medical information on the monitor 47. As an example, the ophthalmologic image processing device 40 according to the present embodiment can generate an Enface image 61, a three-dimensional tomographic image 62, a two-dimensional tomographic image 63, disease information 64, a specific layer image 67, a chart 68, a thickness map (a first map) 69, and normal eye comparison map (a second map) 70 as multiple types of medical information. Also, in the process of generating medical information, segmentation result information 66 is generated. Note that some parts of the multiple medical information generation processes described below are categorized as an “other-information use process” that is performed using medical information generated by a generation process of other medical information.

The Enface image 61 is a two-dimensional front view of the tissue when viewed in a direction along the optical axis (front direction) of the measurement light. The data of the Enface image 61 can be, for example, accumulation image data in which brightness values are accumulated in the depth direction (Z direction) at each position in the XY direction, or accumulated values of spectral data at each position in the XY direction. In this embodiment, the Enface image 61 is generated as medical information by performing an Enface image generation process on the RAW data 60.

The three-dimensional tomographic image 62 is a three-dimensional image of the tissue of the subject eye (in this embodiment, a retinal tissue). In this embodiment, a three-dimensional tomographic image 62 is formed by arranging multiple two-dimensional tomographic images each corresponding to a respective one of the multiple scan lines 51 in the direction perpendicular to the image. In this embodiment, the three-dimensional tomographic image 62 is generated as medical information by performing a tomographic image generation process on the RAW data 60.

The two-dimensional tomographic image 63 is a two-dimensional tomographic image (a two-dimensional image spreading in the depth direction) of the tissue of the subject eye. The ophthalmologic image processing device 40 in this embodiment can extract and generate any two-dimensional tomographic image 63 included in the image range of the three-dimensional tomographic image 62 and display it on the monitor 47. In other words, in this embodiment, the two-dimensional tomographic image 63 displayed on the monitor 47 is not necessarily limited to the two-dimensional tomographic image 63 corresponding to the scanning line 51 (see FIG. 2). The generation process of the two-dimensional tomographic image 63 is categorized as the other-information using process using the three-dimensional tomographic image generated by the tomographic image generation process as described above. Therefore, the tomographic image generation process is performed prior to the generation process of the two-dimensional tomographic image 63. The ophthalmologic image processing device 40 in this embodiment can display, on the monitor 47, the two-dimensional tomographic image 63 at a position specified by a user. If a position is not specified by the user, the ophthalmologic image processing device 40 displays the two-dimensional tomographic image 63 at a position set as a default (a default position). The default position may include, for example, a position that passes horizontally through the center, in a vertical direction, of the two-dimensional front view and a position that passes vertically through the center, in a horizontal direction, of the two-dimensional front view may be included.

Furthermore, in this embodiment, the ophthalmologic image processing device 40 can generate the two-dimensional tomographic image 63 with a higher quality than the two-dimensional tomographic image generated from the RAW data 60 through the tomographic image generation process, and can display it on the monitor 47. In other words, in this embodiment, a two-dimensional tomographic image extracting process is performed on the three-dimensional tomographic image 62, and then a quality improving process is performed to generate the two-dimensional tomographic image 63 as the medical information. The quality improving process may be, for example, an addition averaging process for multiple two-dimensional tomographic images taken at the same position. In the quality improving process, a mathematical model trained by a machine learning algorithm may be used. In this case, the mathematical model is pre-trained to output an image with improved quality of the input two-dimensional tomographic image (for example, an image with reduced noise). The quality improving process is categorized as the other-information using process using the three-dimensional tomographic image 62 or the two-dimensional tomographic image 63 as described above. Therefore, the generation process of the three-dimensional tomographic image 62 or the two-dimensional tomographic image 63 needs to be performed prior to the quality improving process.

The disease information 64 is information about a disease present in the subject eye. The disease information 64 may be information indicating the possibility that at least one of diseases is present in the subject eye. The disease information 64 may also include information about the position of the disease in the tissue appearing in the ophthalmologic image. In this embodiment, a disease information generation process is performed on at least one ophthalmologic image (for example, at least one of the three-dimensional tomographic image 62, the two-dimensional tomographic image 63, and the Enface image 61), whereby the disease information 64 is generated as the medical information. In other words, the disease information generation process is the other-information using process using at least one of the three-dimensional tomographic image 62, the two-dimensional tomographic image 63, and the Enface image 61. Therefore, at least one of the three-dimensional tomographic image 62, the two-dimensional tomographic image 63, and the Enface image 61 needs to be generated prior to performing the disease information generation process. In the disease information generation process, a mathematical model trained by a machine learning algorithm may be used. In this case, the mathematical model is pre-trained to output the disease information about the tissue appearing in the ophthalmologic image when the ophthalmologic image is input.

The segmentation result information 66 is information indicating detection results of at least one of multiple layers included in the tissue appearing in the ophthalmologic image and boundaries between multiple layers (hereinafter, collectively referred to as “layers/boundaries”). In this embodiment of the segmentation result information 66, at least the detection result of an ILM (internal limiting membrane), and the boundary between a RPE (retinal pigment epithelium) and a BM (Bruch's membrane) (RPE/BM) are included. In this embodiment, the segmentation process is performed on the three-dimensional tomographic image 62 (multiple two-dimensional tomographic images constituting the three-dimensional tomographic image 62 may also be used), whereby the segmentation result information 66 is generated. In the segmentation process, a mathematical model trained by a machine learning algorithm may be used. In this case, the mathematical model is pre-trained to output the detection result of at least one of the layers/boundaries appearing in the ophthalmologic image when the ophthalmologic image is input. Furthermore, the CPU 41 may generate the segmentation result information 66 by performing a known image process on the ophthalmologic image. The segmentation process is the other-information using process using the three-dimensional tomographic image 62 (multiple two-dimensional tomographic images constituting the three-dimensional tomographic image 62 may also be used). Therefore, the generation process of the three-dimensional tomographic image 62 needs to be performed prior to performing the segmentation process.

The specific layer image 67 is an image (a three-dimensional image in this embodiment) of a specific layer included in the tissue appearing in the ophthalmologic image. In the specific layer image generation process of this embodiment, a specific layer image generation process is performed to extract a specific layer from the three-dimensional tomographic image 62 based on the segmentation result information 66. As a result, in the specific layer image generation process, the specific layer image 67 is generated as the medical information. In other words, the specific layer image generation process is the other-information using process using the segmentation result information 66 generated by the segmentation process. Therefore, the segmentation process needs to be performed prior to performing the specific layer image generation process.

The chart 68 shows a condition of a specific layer/boundary in each of multiple regions set in the tissue appearing in the ophthalmologic image. In this embodiment, a thickness chart and a volume chart are used as the chart 68. The thickness chart is a chart that shows an average thickness of a specific layer/boundary (for example, a layer/boundary from ILM to RPE/BM) for each region. The volume chart is a chart that shows the average volume of a specific layer for each region. In the chart generation process according to the present embodiment, a specific layer is extracted from the three-dimensional tomographic image 62 based on the segmentation result information 66. Then, by calculating the average thickness or volume of the extracted layer for each region, the chart 68 is generated as the medical information. In other words, the chart generation process is the other-information using process using the segmentation result 66 generated by the segmentation process. Therefore, the segmentation process needs to be performed prior to performing the chart generation process.

The thickness map 69 shows a two-dimensional distribution of the thickness of the specific layer/boundary when viewed from the front side (in a direction along the optical axis of the measurement light) of the tissue appearing in the ophthalmologic image. In the thickness map generation process according to the present embodiment, a specific layer is extracted from the three-dimensional tomographic image 62 based on the segmentation result information 66. Then, by obtaining a two-dimensional distribution of the thickness of the extracted layer, the thickness map 69 is generated as the medical information. In other words, the thickness map generation process is the other-information using process using the three-dimensional tomographic image 62 and the segmentation result 66. Therefore, the three-dimensional tomographic image 62 and the segmentation result 66 needs to be generated prior to performing the thickness map generation process.

The normal eye comparison map 70 shows a comparison result between the thickness map of a normal eye (for example, average data of thickness maps of multiple normal eyes without disease) and the thickness map 69 of the subject eye. In this embodiment, a percentile map and a deviation map are used as the normal eye comparison map 70. The percentile map shows a two-dimensional distribution of the percentile of the difference between the thickness map of the normal eye and the thickness map 69 of the subject eye. The deviation map shows a two-dimensional distribution of deviations between the thickness map of the normal eye and the thickness map 69 of the subject eye. In this embodiment, by performing a normal eye comparison map generation process on the thickness map of the normal eye and the thickness map 69 of the subject eye, the normal eye comparison map 70 is generated as the medical information. In other words, the normal eye comparison map generation process is the other-information using process using the segmentation result 66 generated by the segmentation process. Therefore, the segmentation process needs to be performed prior to performing the normal eye comparison map generation process.

In this embodiment, all the medical information generation processes are executed by the ophthalmologic image processing device 40. However, before the multiple medical information generation processes are executed by the ophthalmologic image processing device 40, one or more types of the medical information may have been generated and saved by another device (for example, the ophthalmologic imaging device 1). For example, among the multiple types of the medical information exemplified in FIG. 3, at least one of the Enface image 61, the three-dimensional tomographic image 62, and the two-dimensional tomographic image 63 may have been previously generated by the ophthalmologic imaging device 1. The detail will be described later.

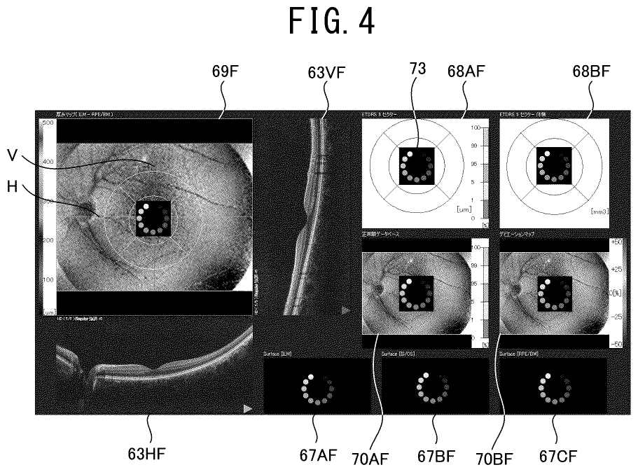

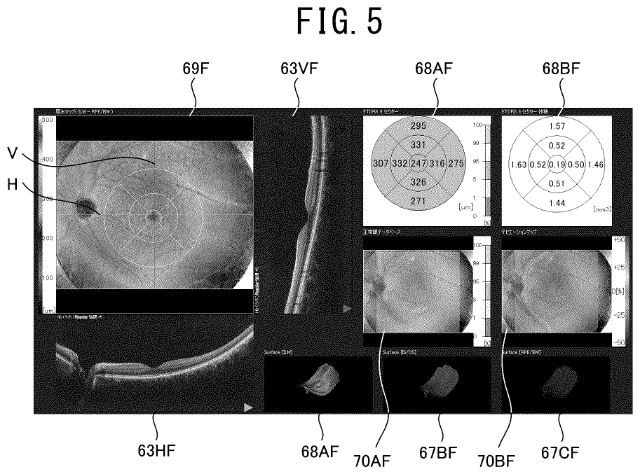

Referring to FIGS. 4 and 5, an overview of a method for displaying the medical information by the ophthalmologic image processing device 40 in this embodiment will be described. FIG. 4 is an example of a display screen of the monitor 47 when one or more types of the medical information about a subject eye is being generated. As shown in FIG. 4, the ophthalmologic image processing device 40 in this embodiment displays multiple display frames each defined for a respective one of multiple types of the medical information on the monitor 47 while the medical information is being generated. As shown in FIGS. 4 and 5, the ophthalmologic image processing device 40 sequentially displays each type of the medical information in the corresponding display frame among the multiple display frames displayed on the monitor 47 each time the corresponding medical information is generated.

In the example shown in FIGS. 4 and 5, the two-dimensional tomographic image 63 at the position of a vertical line V shown in a front view (in this embodiment, a thickness map) is displayed in the display frame 63VF. In other words, the two-dimensional tomographic image 63 extending from the position of the vertical line V in the depth direction of the tissue is displayed in the display frame 63VF. The two-dimensional tomographic image 63 at the position of a horizontal line H shown in the front view (in this embodiment, a thickness map) is displayed in the display frame 63HF That is, the two-dimensional tomographic image 63 extending from the position of the horizontal line H in the depth direction of the tissue is displayed in the display frame 63HF. Note that the front image showing the position of the two-dimensional tomographic image 63 (in this embodiment, the vertical line V and the horizontal line H) may be a front image other than the thickness map. For example, the Enface image 61 or a front image taken by a capturing device different from the ophthalmologic imaging device 1 may be displayed.

In the display frame 67AF, the specific layer image 67 of the ILM (internal limiting membrane) is displayed. In the display frame 67BF, the specific layer image 67 of the IS/OS (junction between photoreceptor inner and outer segments) is displayed. In the display frame 67CF, the specific layer image 67 of the RPE (retinal pigment epithelium) and BM (Bruch's membrane) is displayed.

In the display frame 68AF, the thickness chart 68 showing the average thickness of the specific layer/boundary (in this embodiment, a layer/boundary from ILM to RPE/BM) for each region is displayed. In the display frame 68BF, the volume chart 68 showing the average volume of a specific layer/boundary (in this embodiment, a layer/boundary from ILM to RPE/BM) for each region is displayed.

In the display frame 69F, the thickness map 69 showing the two-dimensional distribution of the thickness of the specific layer (in this embodiment, the layer/boundary from ILM to RPE/BM) when the tissue appearing in the ophthalmologic image is viewed from the front is displayed. As mentioned above, in this embodiment, the vertical line V and the horizontal line H each indicating the position of the two-dimensional tomographic image 63 displayed on the monitor 47 are shown on the thickness map 69.

In the display frame 70AF, the normal eye comparison map (i.e., a percentile map) 70 showing the two-dimensional distribution of the percentile difference between the thickness map of a normal eye and the thickness map 69 of the subject eye is displayed. In the display frame 70BF, a normal eye comparison map (a deviation map) 70 showing the two-dimensional distribution of the deviation between the thickness map of a normal eye and the thickness map 69 of the subject eye is displayed.

Note that the method for displaying the medical information shown in FIGS. 4 and 5 is one example. For example, at least some of the multiple medical information displayed in FIG. 5 may be omitted. In addition, medical information (for example, at least one of the Enface image 61 and the three-dimensional tomographic image 62 shown in FIG. 3) different from the medical information shown in FIG. 5 may be displayed on the monitor 47.

Referring to FIGS. 6 to 12, an explanation will be given about ophthalmologic image processing in this embodiment. In the present embodiment, the ophthalmologic image processing device 40, which is a PC, acquires data of an ophthalmologic image from the ophthalmologic imaging device 1. Then, various types of the medical information are generated by processing the acquired ophthalmologic image data. However, as mentioned above, other devices may also be used as an ophthalmologic image processing device. For example, the ophthalmologic imaging device 1 itself may perform the ophthalmologic image processing. Alternatively, multiple control units (for example, the CPU 31 of the ophthalmologic imaging device 1 and the CPU 41 of the ophthalmologic image processing device 40) may cooperatively perform the ophthalmologic image processing. The CPU 41 of the ophthalmologic image processing device 40 performs the ophthalmologic image processing shown in FIG. 6 according to the ophthalmologic image processing program stored in the NVM 44.

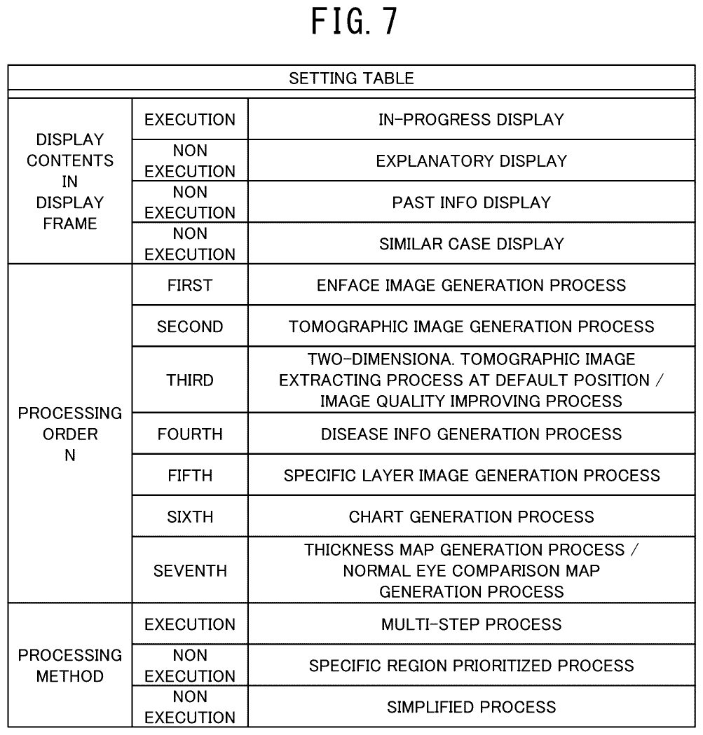

The CPU 41 executes a display frame pre-setting process (S1), a processing order setting process (S2), and a processing method setting process (S3) while waiting for processing of the ophthalmologic image. Until processing of the ophthalmologic image is started (S5: NO), the steps of S1 to S3 are repeated. In this embodiment, settings set in S1 to S3 are stored in a setting table (refer to FIG. 7).

In the display frame pre-setting process (S1), the CPU 41 sets a display content in each display frame during generation of each of types of the medical information according to instructions input by the user (e.g., instructions input via the operation unit 48). As shown in FIG. 7, during the display frame pre-setting process (S1) in this embodiment, the user selects whether to execute each of in-progress display, explanatory display, past information display, and similar case display.

When the in-progress display is executed, the CPU 41 adds an in-progress display image 73 to each display frame as shown in FIG. 4. The in-progress display image 73 indicates that the medical information corresponding to the display frame is currently being generated until the medical information is generated and displayed. In this case, the user can easily grasp that the medical information corresponding to the display frame (i.e., the medical information to be displayed in the display frame) is being generated. As an example, in this embodiment, a progress indicator indicating that the medical information is being generated is added to the display frame as the in-progress display image 73. The in-progress display image 73 may be added inside the display frame or at an outside position adjacent to the display frame. The in-progress display image 73 may be added only to some of the multiple display frames.

When the explanatory display is executed, the CPU 41 adds an explanatory display image to each display frame while multiple types of the medical information are being generated and displayed. The explanatory display image provides an explanation of the medical information corresponding to each display frame. In this case, the user can understand the explanation of the medical information corresponding to the display frame before the actual medical information is displayed. Specific contents of the explanatory display image can be selected as appropriate. For example, as an explanation of the type (category) of the medical information, the messages of “It shows the distribution of the difference of the thicknesses from ILM to RPE/BM between a normative-database and the subject eye” may be provided. As an explanation of how to interpret the medical information, “A darker color indicates a larger difference” may be provided. The explanatory display image may be added only to some of the multiple display frames.

When the past information display is executed, the CPU 41 displays previously-generated medical information in the corresponding display frame until multiple types of the medical information are generated and displayed. The previously-generated information refers to the medical information that was previously generated for the same subject eye as the subject eye for which the ophthalmologic image is currently captured. In this case, the user can grasp the past medical information for the same subject eye until the medical information is generated and displayed in each display frame. As an example, in this embodiment, the CPU 41 identifies the subject eye based on the patient's name or ID. Then, the CPU 41 retrieves the past medical information for the identified subject eye from a storage device (e.g., the NV memory 44) and displays it in each corresponding display frame. The past medical information may be displayed in all the display frames or only in some of the display frames. If no past medical information exists for the subject eye, the process of displaying the past medical information is omitted.

When the similar case display is executed, the CPU 41 identifies one of multiple diseases as a target disease case. The CPU 41 displays the medical information of another subject eye having the identified target disease case as similar case information in the corresponding display frame until the corresponding medical information is generated and displayed. In this case, the user can grasp the similar case until the medical information of the subject eye is displayed. The data of similar cases may be pre-stored in, for example, the NV memory 44. The method for identifying the target case can be selected as appropriate. For example, the CPU 41 may identify a disease specified by the user as the target disease case. Alternatively, the CPU 41 may identify the target disease case based on information about the subject eye. Examples of the information about the subject eye include information about diseases entered in the medical record of the subject eye, information about diseases determined based on past medical information of the subject eye, or information about diseases determined based on images or examination results different from the ophthalmologic images being processed. Additionally, the CPU 41 may identify the target disease case of the subject eye by processing at least a part of the ophthalmologic images by inputting the ophthalmologic images into a mathematical model trained by a machine learning algorithm. Furthermore, the CPU 41 may identify the target disease case based on the type of the ophthalmologic image that is a target for processing. For example, if the ophthalmologic image is an image of an optic disc, the target disease case may be identified as glaucoma. If the ophthalmologic image is a wide-angle image captured with a wider field of view than usual, the target disease case may be identified as at least one of diabetic retinopathy, retinal detachment, and macular hole.

Furthermore, once newly generated medical information based on the processed ophthalmologic image is displayed in the corresponding display frame, the CPU 41 alternately displays the past medical information/the similar case information and the newly generated medical information in the display frame by switching. This switching is performed in response to a user instruction.

In the processing order setting process (S2), the CPU 41 sets an order of executing multiple processes (refer to FIG. 3) to be performed on the ophthalmologic image in response to a user instruction. Therefore, a plurality of types of the medical information are displayed on the monitor 47 in the order that the user wishes to review, making it easier for the use to grasp the medical information more efficiently. As mentioned above, some of the multiple medical information generation processes are the other-information using process using medical information generated by the other-medical information generation process. In this embodiment, the medical information generation process used in the other-information using process is executed prior to executing the other-information using process. For example, when performing the specific layer image generation process, the chart generation process, the thickness map generation process, and the normal eye comparison map generation process, it is assumed that the segmentation result information 66 (refer to FIG. 3) has already been generated. Therefore, the CPU 41 need to complete the segmentation process before starting the specific layer image generation process, the chart generation process, the thickness map generation process, or the normal eye comparison map generation process.

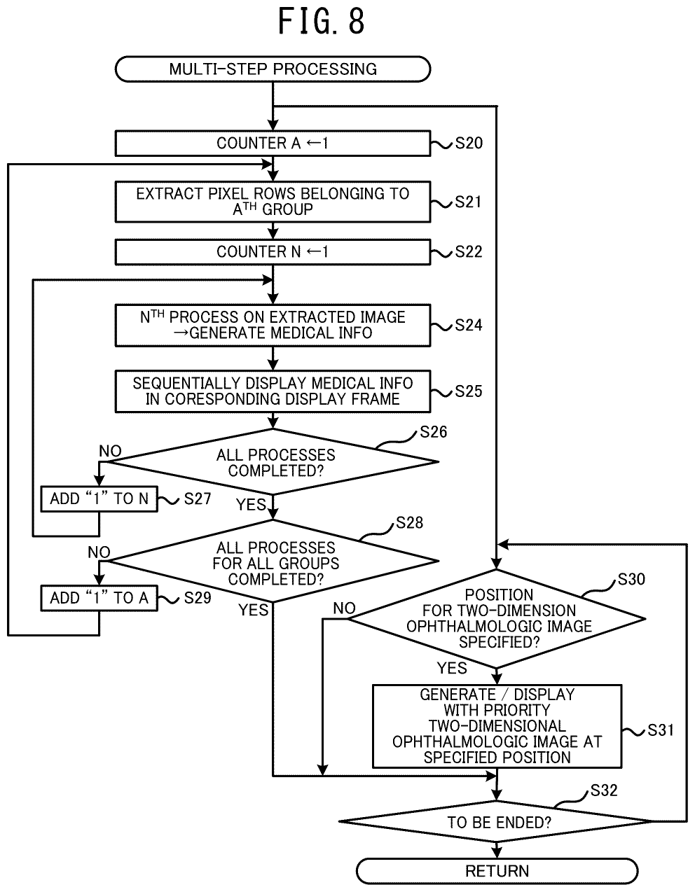

The processing method setting process (S3) selects one of a multi-step process, a specific region prioritized process, and a simplified process, as a processing method, to be executed for processing the ophthalmologic image. The details of each process will be described later with reference to FIGS. 8 to 12.

Returning back to the explanation in FIG. 6. When a trigger to start processing on the ophthalmologic image is input (S5), the CPU 41 acquires the ophthalmologic image of the subject eye to be processed, which is captured by the ophthalmologic imaging device 1 (S6). The CPU 41 displays multiple display frames (refer to FIGS. 4 and 5), each corresponding to one of multiple types of the medical information, on the monitor 47 (S7). The CPU 41 processes the display contents of each of the display frames according to the settings defined at the display frame pre-setting process (S1) (S8). In this embodiment, at least one of the aforementioned in-progress display, explanation display, past information display, and similar case display is executed. Next, the CPU 41 executes one of the multi-step process (S11), specific region prioritized process (S14), or simplified process (S15) according to the settings defined at the processing method setting process (S3).

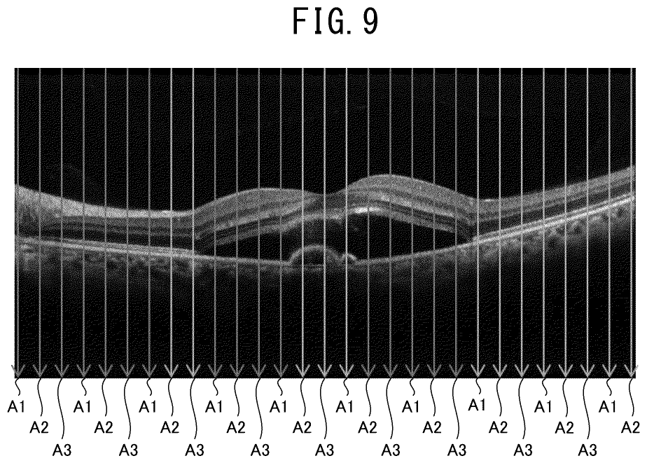

Referring to FIGS. 8 and 9, the multi-step process (S11) will be described. At the multi-step process, a predetermined set of pixels or pixel rows is partially extracted from the entire pixels or pixel rows constituting the ophthalmologic image according to a predetermined rule. At least one of multiple processing steps is executed on the extracted partial image to generate the medical information. The generated medical information is sequentially displayed on the monitor 47. Then, the same process is performed on at least a portion of the remaining image that is not extracted from the entire ophthalmologic image. As a result, the medical information based on the extracted partial images and the remaining image is generated and sequentially displayed on the monitor 47. In other words, at least one of multiple types of medical information is gradually generated and displayed through multiple steps.

As shown in FIG. 8, during the multi-step process, steps (S20-S29) for generating and displaying medical information according to the set processing order and steps (S30-S32) for generating and displaying a two-dimensional tomography image at a specified position are executed in parallel. First, the steps (S20-S29) will be described.

First, the CPU 41 sets the value of counter A to “1” as an initial value (S20). As shown in FIG. 9, the counter A is used to identify the group of pixels or pixel rows to be extracted and processed from among the multiple pixels or pixel rows constituting the ophthalmologic image. In the example shown in FIG. 9, each of multiple pixel rows (a plurality of A-scanned images extending in a depth direction of the tissue in FIG. 9) that constitute the ophthalmologic image (the two-dimensional tomography image in FIG. 9) is regularly classified as “A1, A2, A3, A1, A2, A3, . . . ”. In other words, when K is any natural number (including 0), the “3*K”th pixel row is classified into the “A=1” group, the “(3*K+1)”th pixel row is classified into the “A=2” group, and the “(3*K+2)”th pixel row is classified into the “A=3” group. In this embodiment, the pixels or pixel rows of each group are classified into multiple groups according to a predetermined rule so that multiple pixels or pixel rows belonging to each group have the same interval and the multiple pixels or pixel rows belonging to each group do not overlap with the pixels or pixel rows belonging to other groups. As a result, by extracting pixels or pixel rows belonging to one specific group, multiple pixels or pixel rows are evenly and equidistantly extracted from the entire ophthalmologic image. Note that instead of multiple pixel rows, multiple pixels may be classified according to a predetermined rule. Additionally, if the ophthalmologic image includes multiple two-dimensional images, the CPU 41 may extract the two-dimensional images having multiple pixels and pixel rows as extraction units. The CPU 41 can classify each of the two-dimensional images in the three-dimensional image and extract them at a later process.

The CPU 41 partially extracts the pixels or pixel rows of the Ath group (having an initial value of “1”) from the pixels or pixel rows constituting the entire three-dimensional ophthalmologic image (S21). In the example shown in FIG. 9, when the value of counter A is “1”, multiple pixel rows of “A1” are extracted. When the value of counter A is “2”, multiple pixel rows of “A2” are extracted. When the value of counter A is “3”, multiple pixel rows of “A3” are extracted.

Next, the CPU 41 sets the value of counter N to “1” as an initial value (S22). Counter N is used to identify the order of multiple processing steps (refer to FIG. 2) to be performed on the ophthalmologic image. As mentioned above, the order of multiple processing steps has been set in advance at the processing order setting process (S2 in FIG. 6).

The CPU 41 generates medical information by performing the Nth process on the Ath extracted image obtained at S21 (S24). The CPU 41 sequentially displays the generated medical information in the corresponding display frame (S25). If all the processes for the Ath extracted image are not completed (S26: NO), the value of counter N is incremented by “1” (S27). The process returns to S24, and the next process for the extracted image is performed (S24, S25).

When all the processes for the Ath extracted image are completed (S26: YES), the CPU 41 determines whether all the processes for the extracted images of all groups are completed (S28). If not completed (S28: NO), the value of counter A is incremented by “1” (S29). The process returns to S21, and the process for the extracted images of the next group is performed (S21-S28).

Note that if the value of counter A is 2 or more, at S21, the pixels or pixel rows of the Ath group are extracted from the remaining image that have not been extracted from the ophthalmologic image. At S24, a process is performed on the image extracted this time from the remaining image. Furthermore, at S24, medical information is generated based on the image currently extracted at the process of S21 and the extracted image previously obtained at the process of S21 from the remaining image. In other words, as the value of counter A increases, higher-quality medical information is gradually displayed on the monitor 47 by combining the previously generated medical information and the newly generated medical information. When all the processes for the extracted images of all groups are completed, the process proceeds to S32.

The CPU 41 determines whether the position of the two-dimensional tomographic image 63 to be displayed on the monitor 47 has been specified in the image range of the three-dimensional tomographic image 62 (S30). In this embodiment, the user operates the operation unit 48 to move at least one of the vertical line V and the horizontal line H (shown in FIGS. 4 and 5) on the front image to a desired position. By doing so, the user specifies the position of the two-dimensional tomographic image 63 to be displayed on the monitor 47. If the position of the two-dimensional tomographic image 63 is not specified (S30: NO), the CPU 41 determines whether to end the process (S32). If the process does not end (S32: NO), the process returns to S30, and the steps of S30-S32 are repeated. When the position of the two-dimensional tomographic image 63 is specified (S30: YES), the CPU 41 performs a process to generate the two-dimensional tomographic image 63 at the specified position from the three-dimensional tomographic image 62 and displays it on the monitor 47. This process takes priority over the generation process of other medical information (S20-S29) and is executed first (S31). As a result, the user can promptly check the two-dimensional tomographic image 63 at the specified position.

In the example shown in FIG. 8, all the multiple types of the medical information are generated through multiple steps. However, while some of the multiple types of the medical information may go through multiple steps, it is also acceptable for other of the types of the medical information to be generated through a single step. Additionally, in the example shown in FIG. 8, the multiple generation processes for the multiple types of the medical information are executed in parallel across multiple steps. However, it is also acceptable that after the generation process across multiple steps for one type of the medical information is completed, the generation process of another type of the medical information across multiple steps may be executed.

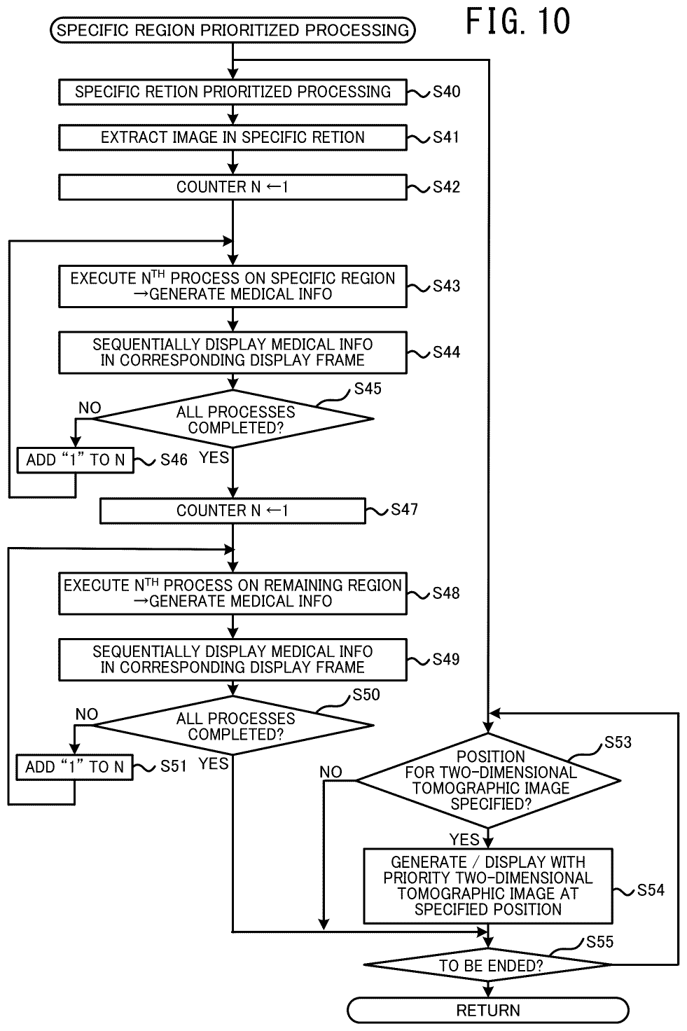

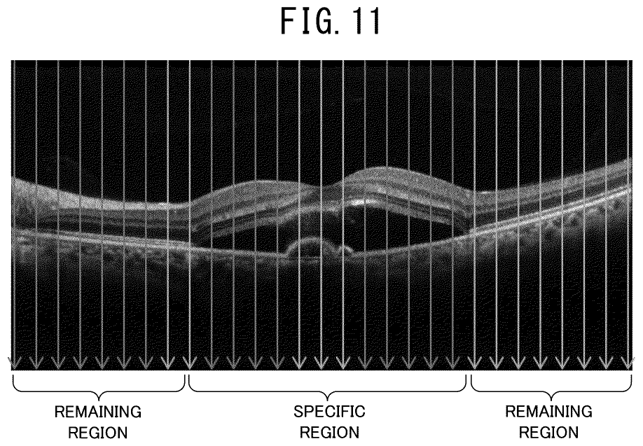

Referring to FIGS. 10 and 11, the specific region prioritized process (S14 in FIG. 6) will be described. In the specific region prioritized process, a part of the image area within the ophthalmologic image is extracted as a specific region (a region of interest). One or more processes for generating the medical information are applied to the extracted image as the specific region, and the generated medical information is sequentially displayed on the monitor 47. Subsequently, the same processes are applied to at least a part of the remaining image area that was not extracted from the ophthalmologic image as the specific region. Accordingly, the medical information based on both the extracted image and the remaining image is generated and sequentially displayed on the monitor 47. In other words, the medical information related to the specific region is generated with higher priority compared to other regions.

As shown in FIG. 10, during the specific region prioritized process, the processing for generating and displaying medical information according to the set processing order (S40-S51) and the processing for generating and displaying the two-dimensional tomographic image at the specified position (S53-S55) are executed in parallel. First, the steps of S40-S51 will be described.

First, the CPU 41 sets the specific region within the three-dimensional image area being processed (S40). For example, the CPU 41 may set the specific region as an image area within the ophthalmologic image where a disease is likely to exist by processing at least a part of the ophthalmologic image. In this case, the CPU 41 may input at least one of the ophthalmologic images into a mathematical model trained by a machine learning algorithm (e.g., a mathematical model trained to output a region where disease is likely to exist) and obtain the region where a disease is likely to exist using the mathematical model. Additionally, the CPU 41 may set the specific region based on information related to the subject eye (e.g., disease information entered in the patient's medical record, disease information determined based on past medical information of the subject eye, or disease information determined based on images or test results that are different from the ophthalmologic image currently being processed). Furthermore, the CPU 41 may set the specific region specified by a user within the ophthalmologic image. In the example shown in FIG. 11, some of pixel columns within the ophthalmologic image (two-dimensional tomographic image in FIG. 9) where a disease is likely to exist are set as the specific region.

The CPU 41 extracts the image within the specific region from the ophthalmologic image currently being processed (S41). Hereafter, the image extracted within the specific region at S41 is referred to as an extracted image, and the image within the remaining region that was not extracted at S41 is referred to as a remaining image. The CPU 41 sets the value of counter N to the initial value of “1” (S42). As mentioned before, counter N is used to determine the order of executing multiple processes (refer to FIG. 2) on the ophthalmologic image.

The CPU 41 generates medical information based on the extracted image by executing the Nth process for the extracted image (S43). The generated medical information is sequentially displayed in the corresponding display frame (S44). If not all processes for the extracted image are not completed (S45: NO), the value of counter N is incremented by “1” (S46), and the process returns to S43. The next sequential process for the extracted image is then executed (S43, S44).

Once all processes for the extracted image are completed (S45: YES), the CPU 41 resets the value of counter N to the initial value of “1” (S47). By executing the Nth process for the remaining image, the CPU 41 generates the medical information based on both the extracted image within the specific region and the remaining image within the remaining region (S48). The generated medical information is sequentially displayed in the corresponding display frame (S49). If all processes for the remaining image are not completed (S50: NO), the value of counter N is incremented by “1” (S51). The process then returns to S48, and the next sequential process for the remaining image is executed (S48, S49). Once all processes for the remaining image are completed (S50: YES), the process transitions to S55.

Next, the process for generating and displaying the two-dimensional tomographic image at the specified position (S53-S55) will be described. The CPU 41 determines whether the position of the two-dimensional tomographic image 63 to be displayed on the monitor 47 is specified within the image area of the three-dimensional tomographic image 62 (S53). If the position of the two-dimensional tomographic image 63 is not specified (S53: NO), the CPU 41 determines whether to end the process (S55). If the process does not end (S55: NO), the steps of S53-S55 are repeated. When the position of the two-dimensional tomographic image 63 is specified (S53: YES), the CPU 41 executes the process of generating the two-dimensional tomographic image 63 at the specified position from the three-dimensional tomographic image 62 and displays it on the monitor 47. This process is given higher priority compared to the generation processes of other medical information (S40-S51).

In the example shown in FIG. 10, the prioritized generation and display of medical information within the specific region are applied to all processes. However, the prioritized process can be applied only to some of the processes. Additionally, in the example shown in FIG. 10, all processes for the extracted image are completed before proceeding to all processes for the remaining image. However, it is also possible to execute, after completing the stepwise generation process of one type of the medical information, the stepwise generation processes for another type of the medical information.

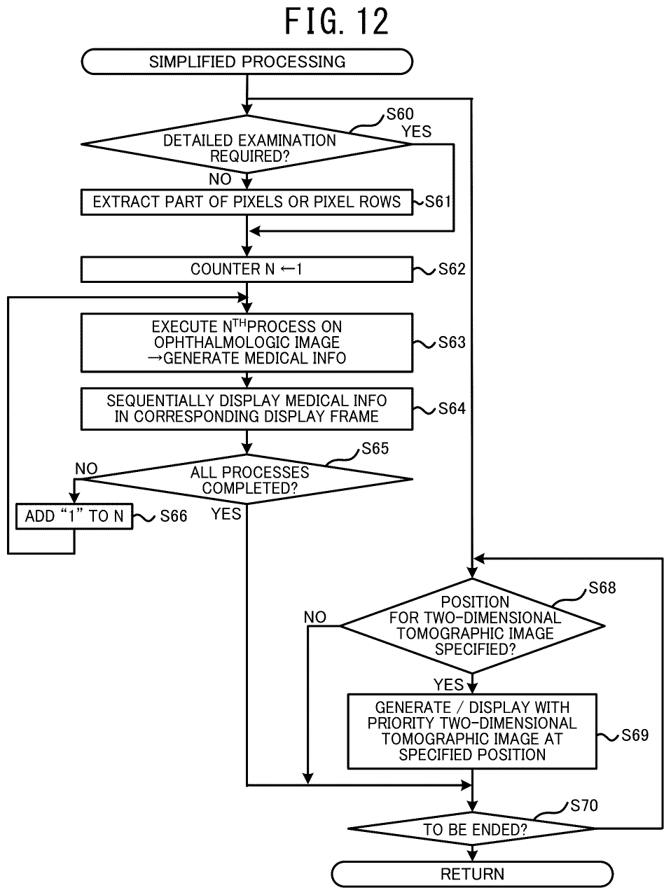

Referring to FIG. 12, the simplified process (S15 in FIG. 6) will be described. In this simplified process, the CPU 41 determines whether a detailed examination on the ophthalmologic image is necessary. For the ophthalmologic image determined to require a detailed examination, the CPU 41 executes the generation process of medical information for the entire ophthalmologic image. For the ophthalmologic image determined not to require a detailed examination, the CPU 41 executes the generation processes of medical information based on the extracted image that was partially extracted from the entire ophthalmologic image. As a result, high-quality medical information is generated for ophthalmologic image that requires a detailed examination, while medical information is generated quickly for the ophthalmologic image that does not require a detailed examination.

As shown in FIG. 12, during the simplified process, processing for generating and displaying medical information according to the set processing order (S60-S66) and processing for generating and displaying the two-dimensional tomographic image at the specified position (S68-S70) are executed in parallel. First, the steps S60-S66 will be described.

First, the CPU 41 determines whether a detailed examination on the target ophthalmologic image is required (S60). For example, the CPU 41 can calculate the probability of a disease existing in the ophthalmologic image by processing at least a portion of the acquired ophthalmologic image. Then, the CPU 41 can determine whether a detailed examination on the ophthalmologic image is required by determining whether the calculated probability exceeds a threshold. In this case, the CPU 41 can input at least a portion of the ophthalmologic image into a machine learning algorithm to obtain the probability of disease existing therein. The mathematical model trained by the machine learning algorithm, for example, may be one that is designed to output the probability of a disease existing therein. The CPU 41 can also obtain the probability of disease existing therein by performing image processing on at least a portion of the ophthalmologic image. Additionally, if information related to the subject eye indicates a high probability of a disease existing, the CPU 41 may determine that a detailed examination on the ophthalmologic image is required. Examples of such information related to the subject eye include disease information entered in the medical record of the subject eye, disease information determined based on past medical information of the subject eye, or disease information determined based on images or examination results different from the ophthalmologic image currently being processed.

If detailed examination is determined not to be necessary (S60: NO), the CPU 41 partially extracts multiple pixels or pixel rows from the entire set of pixels or pixel rows forming the ophthalmologic image according to a predetermined rule (S61). For example, similar to the step shown in S21 of FIG. 8 and FIG. 9, multiple pixels or pixel rows may be uniformly and equidistantly extracted from the entire ophthalmologic image. If a detailed examination is determined to be necessary (S60: YES), the extraction process in S61 is not performed, and the process proceeds directly to S62.

The CPU 41 initializes the value of the aforementioned counter N as “1” (S62). Then, the CPU 41 generates, by executing the Nth processing step for the ophthalmologic image (i.e., the extracted image at S61 or the entire image when no extraction process is performed), medical information (S63). The CPU 41 sequentially displays the generated medical information in the corresponding display frame (S64). If all processes for the ophthalmologic image are not completed (S65: NO), the value of the counter N is incremented by “1” (S66). Then, the process returns to S63, and the next sequential processing for the ophthalmologic image is executed (S63, S64). Once all processes are completed (S65: YES), the process proceeds to S70.

The process for generating and displaying a two-dimensional tomography image at the specified position (S68-S70) will be described. The CPU 41 determines whether the position of the two-dimensional tomography image 63 to be displayed on the monitor 47 has been specified within the image area of the three-dimensional tomography image 62 (S68). If the position of the two-dimensional tomography image 63 is not specified (S68: NO), the CPU 41 determines whether to end the process (S70). If the process is not ended (S70: NO), the steps of S68-S70 are repeated. When the position of the two-dimensional tomography image 63 is specified (S68: YES), the CPU 41 executes the process of generating the two-dimensional tomography image 63 at the specified position from the three-dimensional tomography image 62 and displaying it on the monitor 47. This process takes priority over the generation process of other medical information (S60-S66) (S69).

In the example shown in FIG. 12, when detailed examination is determined not to be necessary, a simplified process (process for the extracted image) is applied to all processes. However, the simplified process may be applied only to a part of the processes.

The disclosed techniques in the above embodiments are merely examples, and therefore, it is possible to modify the techniques described in the above embodiments. It is also possible to omit some of the multiple processes described in the above embodiments. For example, it is possible to omit various displays in the predefined display frames (such as in-progress display, explanation display, past information display, and similar case display).

In the above embodiments, all the generation processes of medical information are performed by the ophthalmologic image processing device 40. However, pre-generated and stored medical information on the target ophthalmologic image (hereinafter, referred to as “pre-generated information”) may have been prepared even before the generation processes of multiple pieces of the medical information by the ophthalmologic image processing device 40 are executed. In this case, regardless of the progress of the generation processes of the medical information based on the target ophthalmologic image (e.g., when generation processes of pieces of the medical information are started), the CPU 41 may display the pre-generated information on the monitor 47. In this case, the user can grasp the pre-generated information displayed on the monitor 47, even before the generation processes of all the medical information are completed. Additionally, the CPU 41 may display the pre-generated information in the display frames each of which corresponds to a respective one of types of pieces of the pre-generated information. In this case, the user can easily grasp the types of the displayed pre-generated information based on the display frames where the information is displayed.

For example, to allow the user to confirm the appropriateness of the captured ophthalmologic image, the ophthalmologic imaging device 1 may generate data of ophthalmologic image to be displayed on the monitor 37 based on RAW data immediately after capturing the images. The ophthalmologic image data may include, for example, Enface images, three-dimensional tomography images, and two-dimensional tomography images. In this case, the CPU 41 of the ophthalmologic image processing device 40 may display at least one of the ophthalmologic images generated by the ophthalmologic imaging device 1 as pre-generated information on the monitor 47 to confirm the appropriateness of the captured results.

The pre-generated information may sometimes be simplified medical information with lower quality or accuracy compared to the same type of medical information generated by the ophthalmologic image processing device 40. In this case, the CPU 41 of the ophthalmologic image processing device 40 may generate the same type of medical information with higher quality or accuracy and display it in the display frame instead of the pre-generated information. In this way, the user can grasp the pre-generated information while waiting for generation of higher quality or accuracy medical information. Once generation of higher quality or accuracy medical information is completed, the user can grasp the newly generated medical information. This allows for a more appropriate understanding of the medical information.

The process of capturing ophthalmologic images at S6 of FIG. 6 is an example of an “image acquisition step.” The processes of generating medical information at the steps of S24 and S31 of FIG. 8, steps of S43, S48, S54 of FIG. 10, and steps S63, S69 of FIG. 12 are examples of “medical information generation steps.” The process of sequentially displaying medical information on the monitor 47 at steps of S25, S31 of FIG. 8, steps of S44, S49, S54 of FIG. 10, and steps of S64, S69 of FIG. 12 are an example of a“sequential display step.” The process of displaying the display frames on the monitor 47 at steps S7, S8 of FIG. 6 is an example of a “frame display step.” The process of identifying the specific disease at step of S8 of FIG. 6 is an example of a“specific disease identification step.” The process of accepting input for specifying the position of the two-dimensional tomography image at steps S30 of FIG. 8, step S53 of FIG. 10, and step S68 of FIG. 12 is an example of a “position specification acceptance step.” The process of partially extracting pixels or pixel columns at steps S21 of FIG. 8 and step S61 of FIG. 12 are an example of an “image extraction step.” The process of determining whether a detailed examination of the ophthalmologic image is necessary at step S60 of FIG. 12 is an example of a “detailed examination determination step.” The process of extracting images within the specific region at steps S40, S41 of FIG. 10 is an example of a “specific region extraction step.” The process of setting the order of multiple processes at step S2 of FIG. 6 is an example of a “process order setting step.”

The above-described embodiments may include the following technical aspect.

One aspect of the present disclosure is a method for processing an ophthalmologic image that is an image of a tissue of a subject eye, the method comprising: acquiring the ophthalmologic image captured by an ophthalmologic imaging device; generating various types of medical information to be displayed on a display unit by performing a plurality of mutually different processes on the acquired ophthalmologic image; and controlling the display unit to sequentially display the various types of medical information upon generating each of the various types of medical information.

1. An ophthalmologic image processing system for processing an ophthalmologic image that is an image of a tissue of a subject eye, the system comprising:
an ophthalmologic imaging device that is configured to capture the ophthalmologic image;
a display unit; and
a control unit that is configured to control the display unit, wherein
the control unit includes at least one processor programmed to:
acquire the ophthalmologic image captured by the ophthalmologic imaging device;
generate various types of medical information to be displayed on the display unit by performing a plurality of mutually different processes on the acquired ophthalmologic image; and
control the display unit to sequentially display the various types of medical information upon generating each of the various types of medical information.

2. An ophthalmologic image processing device for processing an ophthalmologic image that is an image of a tissue of a subject eye, the device comprising:
a control unit including at least one processor programmed to:
acquire the ophthalmologic image captured by an ophthalmologic imaging device;
generate various types of medical information to be displayed on a display unit by performing a plurality of mutually different processes on the acquired ophthalmologic image; and
control the display unit to sequentially display the various types of medical information upon generating each of the various types of medical information.

3. The ophthalmologic image processing device according to claim 2, wherein
the at least one processor is further programmed to:
control the display unit to display a plurality of display frames each of which is defined for a respective one of the various types of medical information,
control the display unit to sequentially display the various types of medical information by displaying each of the various types of medical information in a corresponding one of the plurality of display frames upon generating each of the various types of medical information.

4. The ophthalmologic image processing device according to claim 3, wherein
the at least one processor is further programmed to:
control the display unit to add an in-progress display image or an explanatory display image to each of the plurality of display frames until a corresponding one of the various types of medical information is generated,
the in-progress display image indicates that the corresponding one of the various types of medical information is being currently generated, and
the explanatory display image indicates an explanation on the corresponding one of the various types of medical information.

5. The ophthalmologic image processing device according to claim 3, wherein
the at least one processor is further programmed to:
control the display unit to display at least one of various types of past medical information in at least one of the plurality of display frames until a corresponding one of the various types of medical information is generated, and
the various types of past medical information have been generated on a same subject eye as the subject eye for which the ophthalmologic image is currently captured.

6. The ophthalmologic image processing device according to claim 3, wherein
the at least one processor is further programmed to:
specify at least one of a plurality of diseases as a specific disease, and
control the display unit to display at least one of various types of other subject medical information in at least one of the plurality of display frames until a corresponding one of the various types of medical information is generated, and
the various types of other subject medical information are medical information on another subject suffering from the specific disease.

7. The ophthalmologic image processing device according to claim 2, wherein
the at least one processor is further programmed to:
control the display unit to display a two-dimensional front view image that was captured or generated in advance on a same subject eye as the subject eye for which the ophthalmologic image is currently captured;
generate at least one of a first map indicative of a two-dimensional distribution of thickness of a specific layer and a second map indicative of a comparison of the two-dimensional distribution of thickness of the specific layer between the subject eye and a normal eye; and
control the display unit to display the first map or the second map by superimposing the first map or the second map onto the two-dimensional front view image that is being displayed on the display unit after generating the first map or the second map.

8. The ophthalmologic image processing device according to claim 2, wherein
the at least one processor is further programmed to:
receive an input of an instruction for specifying a position in a three-dimensional tomographic image that is the ophthalmologic image;
generate a two-dimensional tomographic image at the specified position from the three-dimensional tomographic image as one of the various types of medical information upon receiving the input of the instruction for specifying the position.

9. The ophthalmologic image processing device according to claim 2, wherein
the at least one processor is further programmed to:
partially extract a plurality of pixels or pixel rows in accordance with a predetermined rule from entire pixels or pixel rows that form the ophthalmologic image;
generate at least one the various types of medical information by performing at least one of the plurality of processes on an extracted image that is formed of the extracted pixels or pixel rows; and
control the display unit to display the at least one of the various types of medical information generated from the extracted image upon generating each of the at least one of the various types of medical information from the extracted image.

10. The ophthalmologic image processing device according to claim 9, wherein
the at least one processor is further programmed to:
generate at least one of the various types of medical information based on both the extracted image and a remaining image including pixels or pixel rows that were not extracted and left behind by performing the at least one of the plurality of processes on the remaining image after performing the at least one of the plurality of processes on the extracted image; and
display the at least one of the various types of medical information generated based on both the extracted image and the remaining image upon generating each of the at least one of the various types of medical information.

11. The ophthalmologic image processing device according to claim 2, wherein
the at least one processor is further programmed to, if the plurality of mutually different processes include an other-medical information using process during which at least one of the various types of medical information is used:
generate the at least one of the various types of medical information to be used in the other-medical information using process prior to performing the other-medical information using process.

12. A non-transitory, computer readable, storage medium storing an ophthalmologic image processing program for processing an ophthalmologic image that is an image of a tissue of a subject eye, the program, when executed by at least one processor of an ophthalmologic image processing device, causing the at least one processor to perform:
acquiring the ophthalmologic image captured by an ophthalmologic imaging device;
generating various types of medical information to be displayed on a display unit by performing a plurality of mutually different processes on the acquired ophthalmologic image; and
controlling the display unit to sequentially display the various types of medical information upon generating each of the various types of medical information.

13. The ophthalmologic image processing system according to claim 1, wherein
the at least one processor is further programmed to:
control the display unit to display a two-dimensional front view image that was captured or generated in advance on a same subject eye as the subject eye for which the ophthalmologic image is currently captured;
generate at least one of a first map indicative of a two-dimensional distribution of thickness of a specific layer and a second map indicative of a comparison of the two-dimensional distribution of thickness of the specific layer between the subject eye and a normal eye; and
control the display unit to display the first map or the second map by superimposing the first map or the second map onto the two-dimensional front view image that is being displayed on the display unit after generating the first map or the second map.

14. The storage medium according to claim 12, wherein
the program further causes the at least one processor to perform:
controlling the display unit to display a two-dimensional front view image that was captured or generated in advance on a same subject eye as the subject eye for which the ophthalmologic image is currently captured;
generating at least one of a first map indicative of a two-dimensional distribution of thickness of a specific layer and a second map indicative of a comparison of the two-dimensional distribution of thickness of the specific layer between the subject eye and a normal eye; and
controlling the display unit to display the first map or the second map by superimposing the first map or the second map onto the two-dimensional front view image that is being displayed on the display unit after generating the first map or the second map.
